(19) United States
(12) Patent Application Publication
(10) Pub. No.: US 2025/0082953 A1
(43) Pub. Date: Mar. 13, 2025

(54) INTRAORAL PHOTOTHERAPY DEVICE FOR SIMULTANEOUSLY IRRADIATING INNER AND OUTER GINGIVA

(71) Applicant: Taiwan RedEye Biomedical Inc., Hsinchu (TW)

(72) Inventors: Shuo-Ting YAN, Hsinchu City (TW); Chien-Chung TUAN, Hsinchu City (TW); Tsung-Jui LIN, Hsinchu City (TW)

(21) Appl. No.: 18/367,898

(22) Filed: Sep. 13, 2023

(51) Int. Cl. A61N 5/06

(52) U.S. Cl. CPC A61N 5/0603; A61N 5/0613; A61N 2005/0606; A61N 2005/0645; A61N 2005/0663

(57) ABSTRACT

An intraoral phototherapy device for simultaneously irradiating inner and outer gingiva includes a transparent shell and a circuit board. The transparent shell includes a bite portion and a stop portion. The bite portion is mounted on an inner arc surface of the stop portion. The circuit board is mounted in the bite portion, and includes first and second light emitting units. The first and the second light emitting units emit lights toward a first and a second surface of the bite portion. When a user bites the bite portion and turn on the intraoral phototherapy device, the lights emitted by the first and the second light emitting units are emitted to a gum inside and outside upper and lower jaw teeth of the user. Therefore, the intraoral phototherapy device can stimulate an affected part of a gingivitis through a photobiomodulation therapy to achieve an effect of anti-inflammation or alleviating pain.

### BACKGROUND OF THE INVENTION

### 1. Field of the Invention

The present invention relates to a phototherapy device, especially an intraoral phototherapy device for simultaneously irradiating inner and outer gingiva.

### 2. Description of the Related Art

Gingivitis causes symptoms such as redness and swelling of gums, and causes discomfort to teeth. Severe gingivitis may damage tissues around the teeth, which can lead to periodontitis, causing gum recession and tooth loss. Usually, mild gingivitis can be improved by brushing teeth and maintaining good oral hygiene, and the mild gingivitis will heal naturally in about 10-14 days.

However, only brushing teeth or maintaining oral hygiene requires a long recovery time for curing gingivitis, and the gums still have redness and swelling during the recovery time, causing discomfort to the teeth. If a patient seeks clinical help, the dentist usually prescribes anti-inflammatory drugs or painkillers for treatment, but the drugs may have adverse effects on human bodies. Therefore, conventional methods for curing gingivitis of brushing teeth, maintaining good oral hygiene, or taking medication must be improved.

### SUMMARY OF THE INVENTION

In view of the above problems, the present invention provides an intraoral phototherapy device for simultaneously irradiating inner and outer gingiva, which can promote healing of gingivitis through the Photobiomodulation therapy, and can have anti-inflammatory or analgesic effects. Therefore, problems of adverse effects or long recovery time caused by drug treatment can be avoided.

The intraoral phototherapy device for simultaneously irradiating inner and outer gingiva includes a transparent shell and a circuit board.

The transparent shell includes a bite portion and a stop portion. The stop portion includes an inner arc surface and an outer arc surface opposite to the inner arc surface. The bite portion is mounted on the inner arc surface of the stop portion. The bite portion includes a first surface and a second surface opposite to the first surface.

The circuit board is mounted in the bite portion of the transparent shell, and includes a first light emitting surface. The first light emitting surface faces to the first surface of the bite portion. The first light emitting surface of the circuit board includes a first inner arc area and a first outer arc area.

The circuit board further includes multiple first light emitting units. The first light emitting units are mounted on the first light emitting surface, and are separately distributed in the first inner arc area and the first outer arc area. The first inner arc area corresponds to the inner arc surface of the stop portion, and the first outer arc area corresponds to the outer arc surface of the stop portion.

Adenosine triphosphate (ATP) is an important molecule that provides cellular energy, and can be regarded as energy currency of human body cells. The photobiomodulation therapy uses red light or near-infrared light to irradiate mitochondria in cells. When energy of the red light or the near-infrared light is absorbed by cytochrome C oxidase in the mitochondria, the Electron transport chain in the mitochondria can be accelerated to generate more ATP for providing to the cells, such that pain can be relieved, inflammation can be reduced, wound healing can be promoted, and blood circulation can be promoted.

The photobiomodulation therapy has anti-inflammatory or pain-relieving effects, and can promote wound healing without causing damage to the human body. Therefore, when the gums are inflamed, the photobiomodulation therapy can help relieve the pain and discomfort caused by the gingivitis.

The present invention stimulates an affected part of the gingivitis through the photobiomodulation therapy, and light emitted by the first light emitting units simultaneously stimulate the gum tissue of inside and outside of teeth of a user to achieve an effect of repairing or alleviating pain.

When the present invention is in use, the user only needs to bite the bite portion of the transparent shell and turns on the intraoral phototherapy device, so that the light emitted by the first light emitting units mounted on the circuit board can irradiate the gums of inside and outside of the teeth to relieve inflammation and pain of the gums. Then, sore gums, gums atrophy, wound healing, and other discomforts can be soothed.

In addition, the user only needs to bite the intraoral phototherapy device of the present invention, which is a non-invasive treatment, such that the discomfort of the user can be reduced, and medication is not required. Therefore, adverse effects of the medication on the human bodies can be also avoided.

### BRIEF DESCRIPTION OF THE DRAWINGS

FIG. 1 is a schematic diagram of an intraoral phototherapy device for simultaneously irradiating inner and outer gingiva of the present invention;

FIG. 2A is a top view of a schematic perspective diagram of the intraoral phototherapy device for simultaneously irradiating inner and outer gingiva of the present invention;

FIG. 2B is a bottom view of a schematic perspective diagram of the intraoral phototherapy device for simultaneously irradiating inner and outer gingiva of the present invention;

FIG. 3 is a schematic cross sectional diagram of the intraoral phototherapy device for simultaneously irradiating inner and outer gingiva of the present invention;

FIG. 4 is a schematic diagram of a use state of the intraoral phototherapy device for simultaneously irradiating inner and outer gingiva of the present invention;

FIG. 5 is another schematic diagram of the intraoral phototherapy device for simultaneously irradiating inner and outer gingiva of the present invention;

FIG. 6 is a schematic side diagram of the intraoral phototherapy device for simultaneously irradiating inner and outer gingiva of the present invention;

FIG. 7 is a schematic diagram of the appearance of a first embodiment of the intraoral phototherapy device for simultaneously irradiating inner and outer gingiva of the present invention;

FIG. 8 is a block diagram of the first embodiment of the intraoral phototherapy device for simultaneously irradiating inner and outer gingiva of the present invention;

FIG. 9 is a block diagram of a second embodiment of the intraoral phototherapy device for simultaneously irradiating inner and outer gingiva of the present invention.

### DETAILED DESCRIPTION OF THE INVENTION

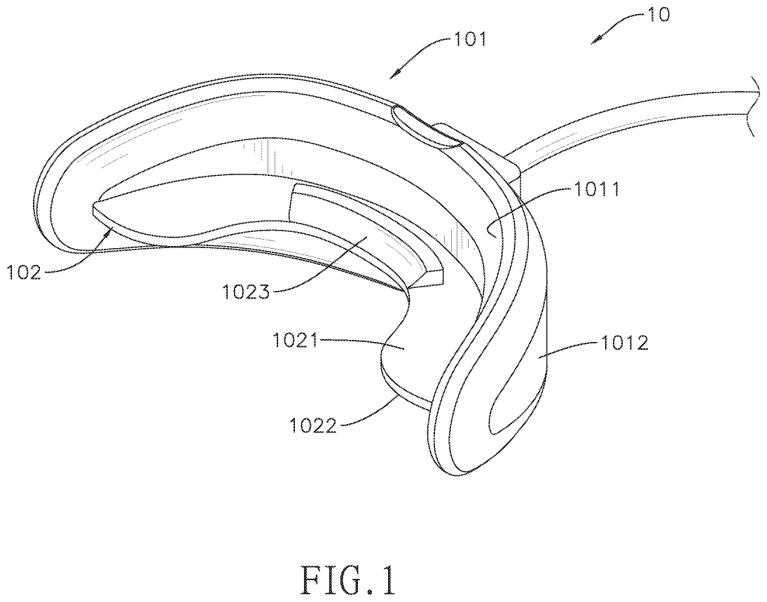

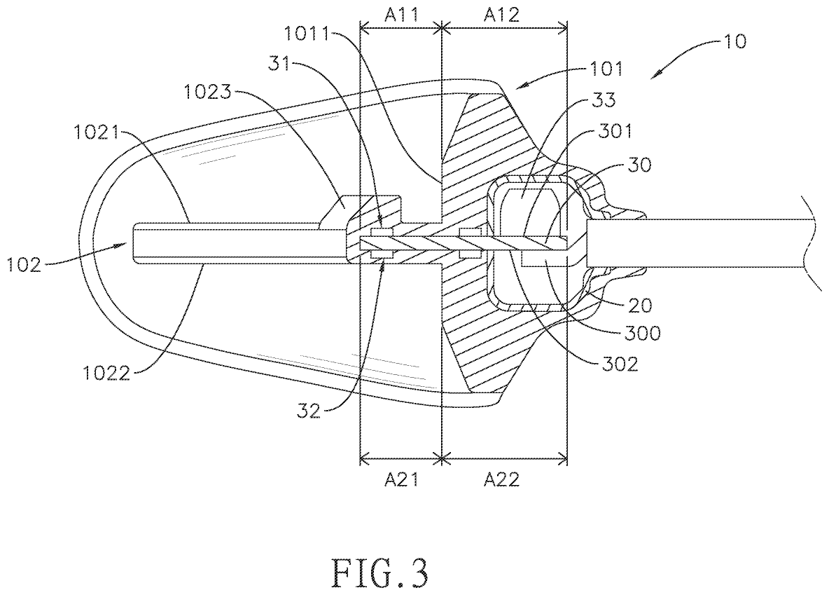

With reference to FIGS. 1, 2A, 2B, and 3, the present invention is an intraoral phototherapy device for simultaneously irradiating inner and outer gingiva, and the intraoral phototherapy device for simultaneously irradiating inner and outer gingiva includes a transparent shell 10, a protective case 20, and a circuit board 30.

The transparent shell 10 includes a stop portion 101 and a bite portion 102. The stop portion 101 includes an inner arc surface 1011 and an outer arc surface 1012 opposite to the inner arc surface 1011. The bite portion 102 is mounted on the inner arc surface 1011 of the stop portion 101. The bite portion 102 includes a first surface 1021 and a second surface 1022 opposite to the first surface 1021.

The protective case 20 is mounted in the stop portion 101 of the transparent shell 10, and includes a circuit board mounting opening for insertion of the circuit board 30. The protective case 20 covers a connection portion of the circuit board 30 and an electric wire 300 to protect the connection portion. In the embodiment, the protective case 20 is a plastic component.

The circuit board 30 is mounted in the bite portion 102 of the transparent shell 10, and includes a first light emitting surface 301 and a second light emitting surface 302 opposite to the first light emitting surface 301. The first light emitting surface 301 faces to the first surface 1021 of the bite portion 102, and the first light emitting surface 301 includes a first inner arc area A11 and a first outer arc area A12. The second light emitting surface 302 faces to the second surface 1022 of the bite portion 102, and the second light emitting surface 302 includes a second inner arc area A21 and a second outer arc area A22. The first inner arc area A11 and the second inner arc area A21 respectively correspond to the inner arc surface 1011 of the stop portion 101. The first outer arc area A12 and the second outer arc area A22 respectively correspond to the outer arc surface 1012 of the stop portion 101. Preferably, when a user bites the bite portion 102, the first inner arc area A11 corresponds to the gum inside upper jaw teeth of the user, the second inner arc area A21 corresponds to the gum inside lower jaw teeth of the user, the first outer arc area A12 corresponds to the gum outside the upper jaw teeth of the user, and the second outer arc area A22 corresponds to the gum outside the lower jaw teeth of the user.

The circuit board 30 further includes multiple light emitting units 31 and multiple light emitting units 32. The first light emitting units 31 are mounted on the first light emitting surface 301 of the circuit board 30, and are separately distributed in the first inner arc area A11 and the first outer arc area A12. The second light emitting units 32 are mounted on the second light emitting surface 302 of the circuit board 30, and are separately distributed in the second inner arc area A21 and the second outer arc area A22. The first light emitting units 31 and the second light emitting units 32 emit lights having wavelengths of 600-1000 nanometers (nm). In this embodiment, the first light emitting units 31 and the second light emitting units 32 may be laser units, red light emitting diodes (LED), or infrared LED units. Preferably, the first light emitting unit 31 may include the laser units 311 and the red LEDs 312, and the laser units 311 and the red LEDs 312 are alternately mounted on the first light emitting surface 301 or the second light emitting surface 302.

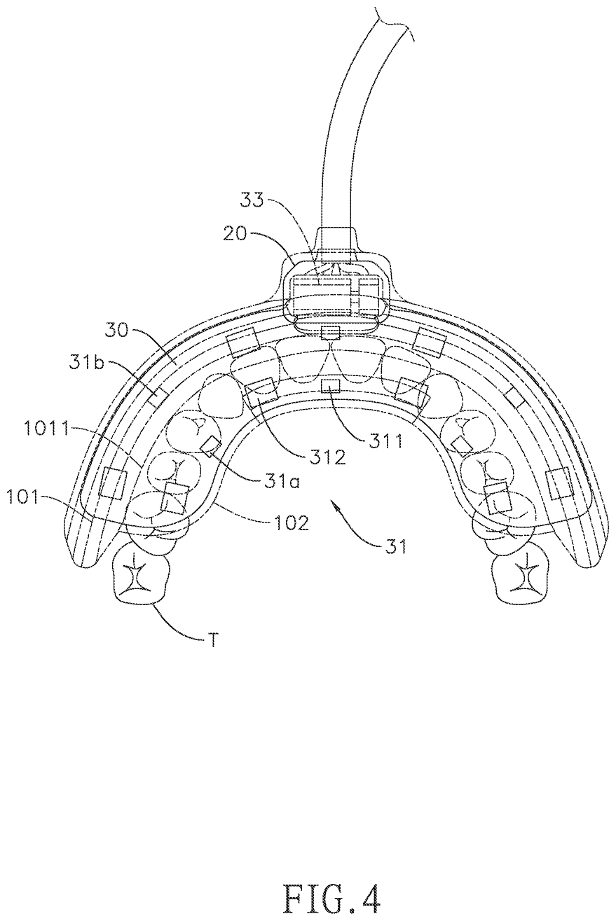

With reference to FIG. 4, when the user bites the bite portion 102 by the teeth T, the first light emitting units 31a mounted in the first inner arc area A11 can emit light to the gum inside the upper jaw teeth of the user, and the first light emitting units 31b mounted in the first outer arc area A12 can emit light to the gum outside the upper jaw teeth of the user. Similarly, the second light emitting units 32 mounted in the second inner arc area A21 can emit light to the gum inside the lower jaw teeth of the user, and the second light emitting units 32 mounted in the second outer arc area A22 can emit light to the gum outside the lower jaw teeth of the user. Namely, the intraoral phototherapy device of the present invention can simultaneously emit lights to the gums inside and outside the teeth to achieve a more comprehensive irradiation state. Then, sore gums, gums atrophy, wound healing, and other discomforts can be further soothed.

Moreover, since the bite portion 102 is mounted on the inner arc surface 1011 of the stop portion 101, when a user bites the bite portion 102, the teeth T of the user can be stopped by the inner arc surface 1011 of the stop portion 101, and the first surface 1021 of the bite portion 102 can directly face to the gum of the upper jaw teeth of the user.

At the same time, the second surface 1022 of the bite portion 102 can directly face to the gum of the lower jaw teeth of the user. Therefore, when the user bites the bite portion 102, the first light emitting units 31 mounted on the circuit board 30 can directly emit lights to the gum of the upper jaw teeth of the user, and the second light emitting units 32 mounted on the circuit board 30 can directly emit lights to the gum of the lower jaw teeth of the user, such that sore gums, gums atrophy, wound healing, and other discomforts of the user can be soothed.

For example, the first inner arc area A11 and the first outer arc area A12 are separated by the inner arc surface 1011 of the stop portion 101. The second inner arc area A21 and the second outer arc area A22 are also separated by the inner arc surface 1011 of the stop portion 101. When the user bites the bite portion 102, the teeth of the user can be stopped by the stop portion 101, the first inner arc area A11 and the second inner arc area A21 can correspond to the gum inside the teeth of the user, and the first outer arc area A12 and the second outer arc area A22 can correspond to the gum outside the teeth of the user.

In this embodiment, the transparent shell 10 is made of a soft material. For example, the transparent shell 10 is made of silicone rubber, so that the user can directly bite the transparent shell 10, and the silicone rubber can provide a more comfortable feel of bite. At the same time, the silicone rubber has a waterproof function, such that the circuit board 30 mounted in the transparent shell 10 can be protected from moisture.

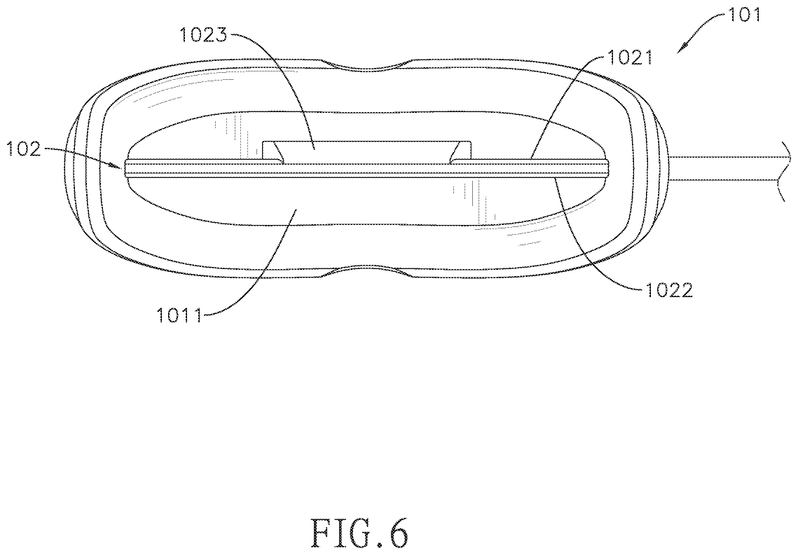

With reference to FIGS. 1, 3, 5, and 6, the bite portion 102 of the transparent shell 10 is flat. The first surface 1021 and the second surface 1022 of the bite portion 102 are respectively perpendicular to the inner arc surface 1011 of the stop portion 101.

Therefore, the user can directly bite the flat bite portion 102 with teeth, and the bite portion 102 can allow the user to easily bite for improving comfort.

Moreover, the first surface 1021 of the bite portion 102 protrudes a positioning block 1023. When the user bites the bite portion 102, the teeth of the user can be positioned between the positioning block 1023 and the inner arc surface 1011 of the stop portion 101, such that a position of the teeth of the user can be fixed. Then, the first light emitting units 31 mounted in the first inner arc area A11 and the first outer arc area A12 can precisely emit lights to the gum inside and outside of the upper jaw teeth of the user, and the second light emitting units 32 mounted in the second inner arc area A21 and the second outer arc area A22 can precisely emit lights to the gum inside and outside of the lower jaw teeth of the user.

Furthermore, the stop portion 101 of the transparent shell 10 is an arc plate. Since the stop portion 101 is the arc plate, when the user bites the bite portion 102, the inner arc surface 1011 of the stop portion 101 can more fittingly contact the gum of the user according to the same shape, such that the position of the teeth of the user can be easily fixed.

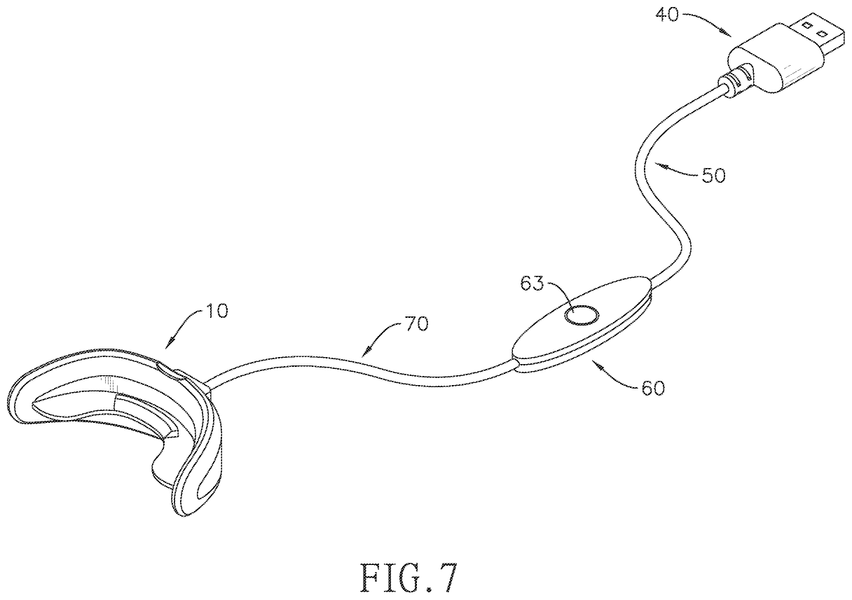

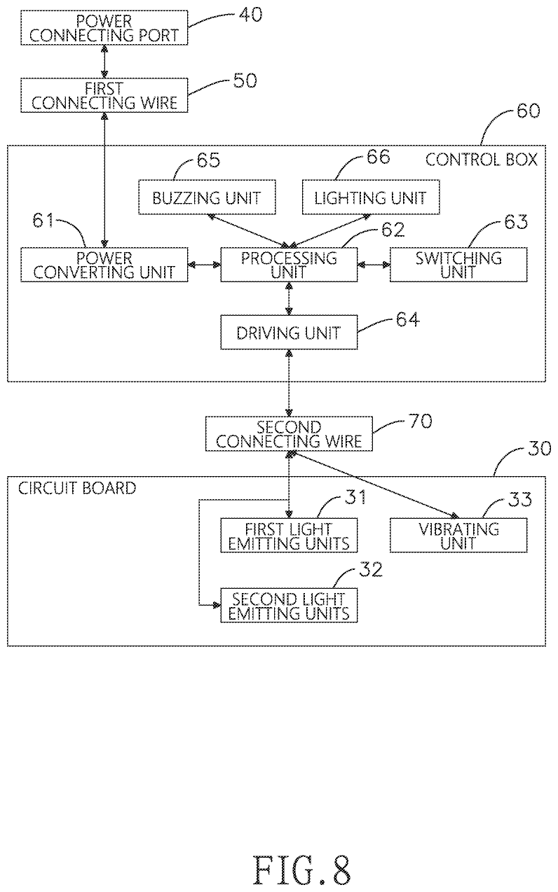

With reference to FIGS. 7 and 8, in a first embodiment of the present invention, the intraoral phototherapy device for simultaneously irradiating inner and outer gingiva further includes a power connecting port 40, a first connecting wire 50, a control box 60, and a second connecting wire 70.

The control box 60 includes a power converting unit 61, a processing unit 62, a switching unit 63, a driving unit 64, a buzzing unit 65, and a lighting unit 66.

The power converting unit 61 connects to the power connecting port 40 through the first connecting wire 50. The power converting unit 61 receives an external electricity provided by an external power, and converts the external electricity to a powering electricity. In this embodiment, the power connecting port 40 is a universal serial bus (USB) male connecting port for connecting to a power adaptor, and the power connecting port 40 receives the external electricity with 5 volts (V) and 1 ampere (A).

The processing unit 62 is electrically connected to the power converting unit 61 for receiving the powering electricity.

The switching unit 63 is electrically connected to the processing unit 62. When the switching unit 63 generates a starting signal, the processing unit 62 receives the starting signal, and generates a light controlling signal according to the starting signal.

The driving unit 64 is electrically connected to the processing unit 62. When the driving unit 64 receives the light controlling signal, the driving unit 64 generates a light driving signal according to the light controlling signal.

The buzzing unit 65 is electrically connected to the processing unit 62. When the switching unit 63 generates the starting signal, the processing unit 62 receives the starting signal, and further generates a reminding signal according to the starting signal. When the buzzing unit 65 receives the reminding signal, the buzzing unit 65 generates a reminding audio according to the reminding signal.

The lighting unit 66 is electrically connected to the processing 62. When the switching unit 63 generates the starting signal, the processing unit 62 receives the starting signal, and further generates the reminding signal according to the starting signal. When the lighting unit 66 receives the reminding signal, the lighting unit 66 emits a reminding light according to the reminding signal. In this embodiment, the switching unit 63 is a circular push button, and the lighting unit 66 is an annular light emitting diode (LED) around the switching unit 63. For example, the lighting unit 66 is a RGB LED. When the switching unit 66 is started by being pressed, the lighting unit 66 can emit a colorful light for reminding the user of a current state of the intraoral phototherapy device.

The second connecting wire 70 is electrically connected between the circuit board 30 and the control box 60. One terminal of the second connecting wire 70 is electrically connected to the first light emitting units 31 and the second light emitting units 32 mounted on the circuit board 30, and another terminal of the second connecting wire 70 is electrically connected to the driving unit 64 of the control box 60.

When the driving unit 64 generates the light driving signal, the first light emitting units 31 and the second light emitting units 32 receive the light driving signal through the second connecting wire 70, and emit the light according to the light driving signal.

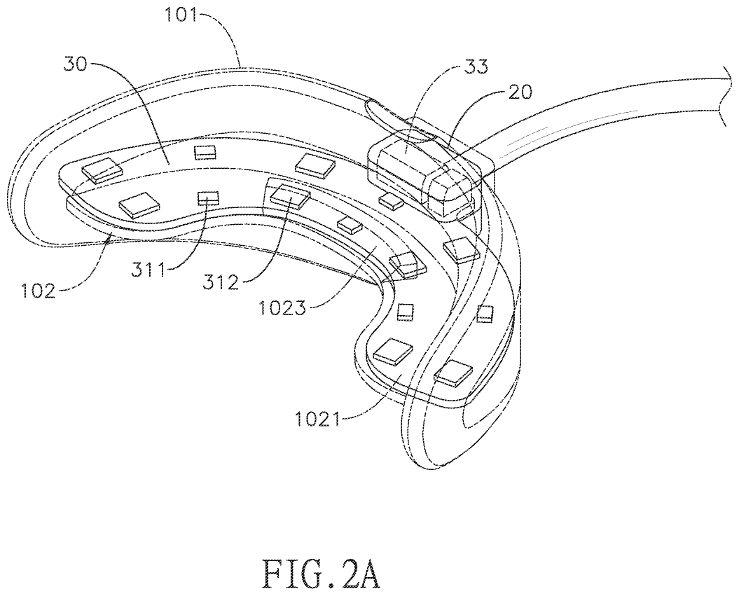

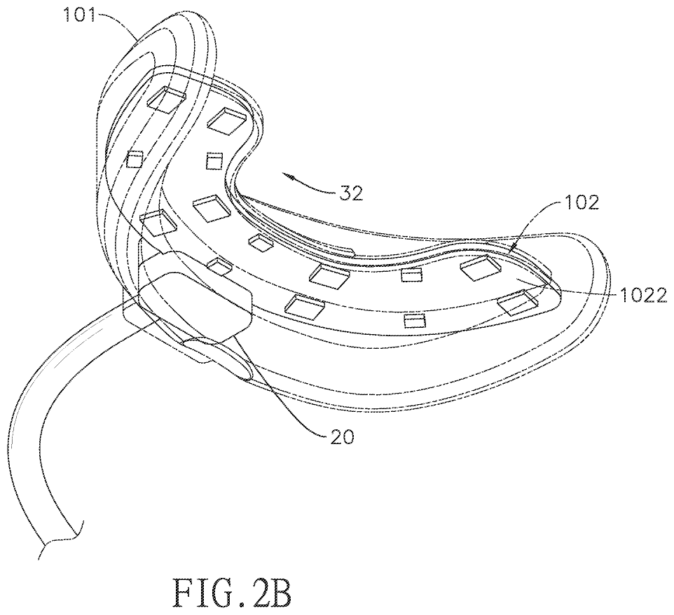

Moreover, with reference to FIGS. 2A, 3, and 4, the circuit board 30 further includes at least one vibrating unit 33. The at least one vibrating unit 33 is mounted on the first light emitting surface 301 or the second light emitting surface 302 of the circuit board 30, and is positioned in the protective case 20. In the embodiment, the at least one vibrating unit 33 is mounted on the first light emitting surface 301, and is positioned in the protective case 20. The second connecting wire 70 is electrically connected to the at least one vibrating unit 33. When the switching unit 63 generates the starting signal, the processing unit 62 receives the starting signal, and further generates a vibration controlling signal according to the starting signal. When the driving unit 64 receives the vibration controlling signal, the driving unit 64 generates a vibration driving signal according to the vibration controlling signal. When the driving unit 64 generates the vibration driving signal, the at least one vibrating unit 33 receives the vibration driving signal through the second connecting wire 70, and generates vibrations according to the vibration driving signal. In another embodiment, the circuit board 30 may include multiple vibrating units 33. The multiple vibrating units 33 are respectively mounted on the first light emitting surface 301 and the second light emitting surface 302, and are both positioned in the protective case 20.

Therefore, when the user uses the intraoral phototherapy device for simultaneously irradiating inner and outer gingiva, not only the first light emitting units 31 and the second light emitting units 32 can emit the light to the gum inside and outside of the upper jaw teeth and the lower jaw teeth of the user for soothing sore gums, gums atrophy, wound healing, and other discomforts of the user, but also the at least one vibrating unit 33 can generate vibration for providing an effect of massage to further sooth discomforts of the gum of the user.

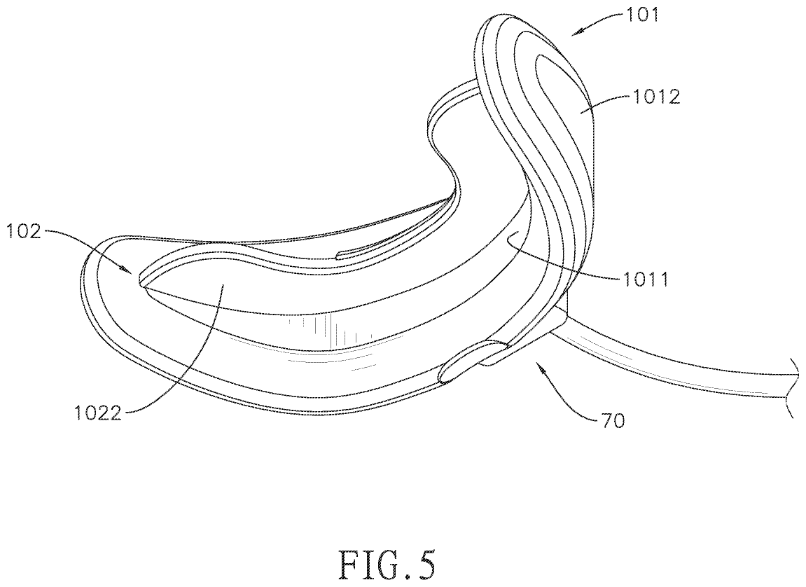

With reference to FIGS. 5 and 7, the second connecting wire 70 extends from the outer arc surface 1012 of the stop portion 101 into the transparent shell 10 to electrically connect to the first light emitting units 31 and the second light emitting units 32 mounted on the circuit board 30.

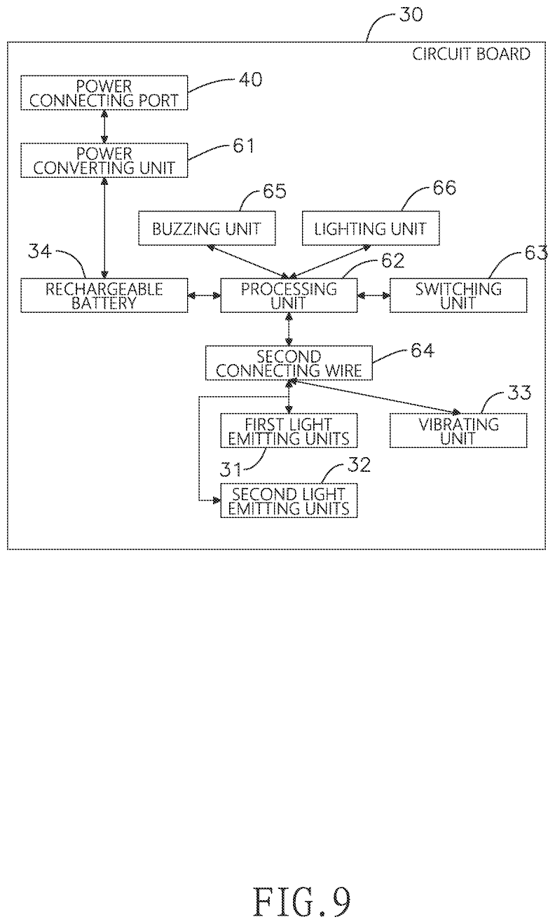

Moreover, with reference to FIG. 9, in a second embodiment of the present invention, the intraoral phototherapy device further includes a power connecting port 40, a power converting unit 61, a processing unit 62, a switching unit 63, a driving unit 64, a buzzing unit 65, a lighting unit 66, and a rechargeable battery 34.

In this embodiment, the power connecting port 40 is mounted on the transparent shell 10. The power converting unit 61 is electrically connected to the power connecting port 40 for receiving an external electricity provided by an external power, and the power converting unit 61 converts the external electricity to a powering electricity. The rechargeable battery 34 is electrically connected to the power converting unit 61 for receiving and storing the powering electricity. The processing unit 62 is electrically connected to the rechargeable battery 34 for receiving the powering electricity through the rechargeable battery 34. In this embodiment, the power connecting port 40 may be a USB female connecting port for inserting a charging wire, and for connecting to a power adaptor through the charging wire.

The switching unit 63 is electrically connected to the processing unit 62. When the switching unit 63 generates a starting signal, the processing unit 62 receives the starting signal, and generates a light controlling signal according to the starting signal.

The driving unit 64 is electrically connected to the processing unit 62, the first light emitting units 31, and the second light emitting units 32. When the driving unit 64 receives the light controlling signal, the driving unit 64 generates a light driving signal according to the light controlling signal. When the driving unit 64 generates the light driving signal, the first light emitting units 31 and the second light emitting units 32 receive the light driving signal, and emits the light according to the light driving signal.

In this embodiment, the circuit board 30 may further include at least one vibrating unit 33. The at least one vibrating unit 33 is mounted on the first light emitting surface 301 or the second light emitting surface 302 of the circuit board 30, and is positioned in the protective case 20. In this embodiment, the at least one vibrating unit 33 is mounted on the first light emitting surface 301, and is positioned in the protective case 20. When the switching unit 63 generates the starting signal, the processing unit 62 receives the starting signal, and further generates a vibration controlling signal according to the starting signal. When the driving unit 64 receives the vibration controlling signal, the driving unit 64 generates a vibration driving signal according to the vibration controlling signal. When the driving unit 64 generates the vibration driving signal, the at least one vibrating unit 33 receives the vibration driving signal, and generates vibrations according to the vibration driving signal. In another embodiment, the circuit board 30 may include multiple vibrating units 33. The multiple vibrating units 33 are respectively mounted on the first light emitting surface 301 and the second light emitting surface 302, and are both positioned in the protective case 20.

The second embodiment of the present invention mounts the rechargeable battery 34 in the transparent shell 10. Therefore, the intraoral phototherapy device for simultaneously irradiating inner and outer gingiva may not need to connect to the external power through a connecting wire, and the intraoral phototherapy device for simultaneously irradiating inner and outer gingiva can be integrated as an independent device without the connecting wire. Namely, when the user uses the intraoral phototherapy device for simultaneously irradiating inner and outer gingiva, the intraoral phototherapy device for simultaneously irradiating inner and outer gingiva can be directly inserted into an oral cavity of the user without opening a mouth of the user to allow the connecting wire to extend out, so that the discomforts of the user can be further soothed.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. An intraoral phototherapy device for simultaneously irradiating inner and outer gingiva, comprising:
a transparent shell, comprising a bite portion and a stop portion; wherein the stop portion comprises an inner arc surface and an outer arc surface opposite to the inner arc surface; wherein the bite portion is mounted on the inner arc surface of the stop portion; wherein the bite portion comprises a first surface and a second surface opposite to the first surface;
a circuit board, mounted in the bite portion of the transparent shell, and comprising a first light emitting surface; wherein the first light emitting surface faces to the first surface of the bite portion; wherein the first light emitting surface comprises a first inner arc area and a first outer arc area;
wherein the circuit board further comprises:
multiple first light emitting units, mounted on the first light emitting surface, and separately distributed in the first inner arc area and the first outer arc area; wherein the first inner arc area corresponds to the inner arc surface of the stop portion, and the first outer arc area corresponds to the outer arc surface of the stop portion.

2. The intraoral phototherapy device for simultaneously irradiating inner and outer gingiva as claimed in claim 1, wherein the circuit board further comprises a second light emitting surface opposite to the first light emitting surface; wherein the second light emitting surface faces to the second surface of the bite portion; wherein the second light emitting surface of the circuit board comprises a second inner arc area and a second outer arc area;
wherein the circuit board further comprises multiple second light emitting units; wherein the second light emitting units are mounted on the second light emitting surface, and separately distributed in the second inner arc area and the second outer arc area; wherein the second inner arc area corresponds to the inner arc surface of the stop portion, and the second outer arc area corresponds to the outer arc surface of the stop portion

3. The intraoral phototherapy device for simultaneously irradiating inner and outer gingiva as claimed in claim 1, wherein the bite portion of the transparent shell is flat; wherein the first surface and the second surface of the bite portion are respectively perpendicular to the inner arc surface of the stop portion.

4. The intraoral phototherapy device for simultaneously irradiating inner and outer gingiva as claimed in claim 1, wherein the first surface of the bite portion protrudes a positioning block.

5. The intraoral phototherapy device for simultaneously irradiating inner and outer gingiva as claimed in claim 2, further comprising:
a power connecting port;
a first connecting wire;
a control box, comprising:
a power converting unit, connecting to the power connecting port through the first connecting wire; wherein the power converting unit receives an external electricity provided by an external power, and converts the external electricity to a powering electricity;
a processing unit, electrically connected to the power converting unit for receiving the powering electricity;
a switching unit, electrically connected to the processing unit; wherein when the switching unit generates a starting signal, the processing unit receives the starting signal, and generates a light controlling signal according to the starting signal;
a driving unit, electrically connected to the processing unit; wherein when the driving unit receives the light controlling signal, the driving unit generates a light driving signal according to the light controlling signal;
a second connecting wire, electrically connected to the first light emitting units and the second light emitting units, and further electrically connected to the driving unit of the control box;
wherein when the driving unit generates the light driving signal, the first light emitting units and the second light emitting units receive the light driving signal through the second connecting wire, and emit light according to the light driving signal.

6. The intraoral phototherapy device for simultaneously irradiating inner and outer gingiva as claimed in claim 5, wherein the second connecting wire extends from the outer arc surface of the stop portion into the transparent shell to electrically connect to the first light emitting units and the second light emitting units mounted on the circuit board.

7. The intraoral phototherapy device for simultaneously irradiating inner and outer gingiva as claimed in claim 5, wherein the circuit board further comprises:
at least one vibrating unit, mounted on the first light emitting surface or the second light emitting surface of the circuit board;
wherein the second connecting wire is further electrically connected to the at least one vibrating unit;
wherein when the switching unit generates the starting signal, the processing unit receives the starting signal, and further generates a vibration controlling signal according to the starting signal;
wherein when the driving unit receives the vibration controlling signal, the driving unit generates a vibration driving signal according to the vibration controlling signal;
wherein when the driving unit generates the vibration driving signal, the at least one vibrating unit receives the vibration driving signal through the second connecting wire, and generates vibrations according to the vibration driving signal.

8. The intraoral phototherapy device for simultaneously irradiating inner and outer gingiva as claimed in claim 5, wherein the control box further comprises:
a buzzing unit, electrically connected to the processing unit;
wherein when the switching unit generates the starting signal, the processing unit receives the starting signal, and further generates a reminding signal according to the starting signal;
wherein when the buzzing unit receives the reminding signal, the buzzing unit generates a reminding audio according to the reminding signal.

9. The intraoral phototherapy device for simultaneously irradiating inner and outer gingiva as claimed in claim 5, wherein the control box further comprises:
a lighting unit, electrically connected to the processing unit;
wherein when the switching unit generates the starting signal, the processing unit receives the starting signal, and further generates the reminding signal according to the starting signal;
wherein when the lighting unit receives the reminding signal, the lighting unit emits a reminding light according to the reminding signal.

10. The intraoral phototherapy device for simultaneously irradiating inner and outer gingiva as claimed in claim 2, wherein the control box further comprises:
a power connecting port, mounted on the transparent shell;
a power converting unit, electrically connected to the power connecting port for receiving an external electricity provided by an external power; wherein the power converting unit converts the external electricity to a powering electricity;
a rechargeable battery, electrically connected to the power converting unit for receiving and storing the powering electricity;
a processing unit, electrically connected to the rechargeable battery for receiving the powering electricity through the rechargeable battery;
a switching unit, electrically connected to the processing unit; wherein when the switching unit generates a starting signal, the processing unit receives the starting signal, and generates a light controlling signal according to the starting signal;
a driving unit, electrically connected to the processing unit, the first light emitting units, and the second light emitting units; wherein when the driving unit receives the light controlling signal, the driving unit generates a light driving signal according to the light controlling signal;
wherein when the driving unit generates the light driving signal, the first light emitting units and the second light emitting units receive the light driving signal, and emits the light according to the light driving signal.
